(19) United States
(12) Patent Application Publication
(10) Pub. No.: US 2008/0050415 A1
(43) Pub. Date: Feb. 28, 2008

(54) Polymeric/ceramic composite materials for use in medical devices

(75) Inventors: Liliana Atanasoska, Edina, MN (US); Michele Zoromski, Minneapolis, MN (US); Robert Warner, Woodbury, MN (US); Steve Kangas, Woodbury, MN (US)

Correspondence Address: MAYER & WILLIAMS PC, 251 NORTH AVENUE WEST, 2ND FLOOR, WESTFIELD, NJ, 07090, US

(73) Assignee: Boston Scientic Scimed, Inc.

(21) Appl. No.: 11/809,906

(22) Filed: Jun. 1, 2007

Related U.S. Application Data
(60) Provisional application No. 60/840,359, filed on Aug. 25, 2006.

(51) Int. Cl. A61F 2/01; A61F 2/04; A61F 2/24; A61K 31/337; A61K 33/24; A61K 38/16; A61P 43/00; C08F 212/08

(52) U.S. Cl. 424/423; 424/649; 514/12; 514/449; 523/105

(57) ABSTRACT

According to an aspect of the invention, implantable or insertable medical devices are provided, which contain one or more composite regions. These composite regions, in turn, contain (a) a polymeric component comprising a vinyl aromatic polymer and (b) a ceramic component comprising a metal or semi-metal oxide.

### STATEMENT OF RELATED APPLICATION

This application claims the benefit of U.S. Provisional Patent Application Ser. No. 60/840,359, filed Aug. 25, 2006, entitled “Polymeric/Ceramic Composite Materials for Use in Medical Devices”, which is incorporated by reference herein.

### FIELD OF THE INVENTION

The present invention relates to new and improved materials for the construction of medical devices.

### BACKGROUND OF THE INVENTION

Various polymer coated medical devices are known which are configured for implantation or insertion into a subject.

Such devices have attendant mechanical requirements, which can be quite demanding. For example, the strength of the polymer coatings, as well as their degree of adhesion to underlying medical device substrates, are important parameters in certain applications.

Devices of this type have also been developed, which deliver therapeutic agents from drug eluting polymer coatings upon implantation or insertion into the body. Specific examples of such devices include drug eluting coronary stents, which are commercially available from Boston Scientific Corp. (TAXUS), Johnson & Johnson (CYPHER), and others. These existing products are based on metallic balloon expandable stents with biostable polymer coatings, which release antiproliferative drugs at a controlled rate and total dose. Specific examples of polymers for drug eluting polymer coatings include block copolymers, such as block copolymers containing polyisobutylene and polystyrene blocks, for instance, polystyrene-polyisobutylene-polystyrene triblock copolymers (SIBS copolymers), which are described in U.S. Pat. No. 6,545,097 to Pinchuk et al. These polymers have proven valuable in implantable and insertable medical devices for a variety of reasons, including their excellent elasticity, strength and biocompatibility. However, the ability to tailor the kinetic drug release (KDR) from these polymers is limited.

### SUMMARY OF THE INVENTION

According to an aspect of the invention, implantable or insertable medical devices are provided, which contain one or more composite regions. These composite regions, in turn, contain (a) a polymeric component comprising a vinyl aromatic polymer and (b) a ceramic component comprising a metal or semi-metal oxide. In certain embodiments, the composite regions further include a therapeutic agent, which is released into a subject.

An advantage of the present invention is that, in certain embodiments, medical devices may be provided with composite regions that provide for enhanced mechanical characteristics, including enhanced strength, toughness and/or abrasion resistance.

Another advantage of the present invention is that, in certain embodiments, medical devices may be provided with composite regions which have improved adhesion to underlying substrate materials.

Yet another advantage of the present invention is that, in certain embodiments, medical devices are provided whose drug eluting properties may be readily tailored.

These and other aspects, embodiments and advantages of the present invention will become immediately apparent to those of ordinary skill in the art upon review of the Detailed Description and Claims to follow.

### DETAILED DESCRIPTION OF THE INVENTION

In one aspect, the present invention provides implantable or insertable medical devices that contain one or more composite regions. These composite regions, in turn, contain (a) a polymeric component comprising a vinyl aromatic polymer and (b) a ceramic component comprising a metal or semi-metal oxide. The polymeric component is preferably a non-extruded polymeric component, and the vinyl aromatic polymer preferably comprises polar groups, ionic groups, or both.

Specific medical devices for use in conjunction with the present invention include a wide variety of implantable or insertable medical devices, which are implanted or inserted either for procedural uses or as implants. Examples include balloons, catheters (e.g., renal or vascular catheters such as balloon catheters), guide wires, filters (e.g., vena cava filters), stents (including coronary artery stents, peripheral vascular stents such as cerebral stents, urethral stents, ureteral stents, biliary stents, tracheal stents, gastrointestinal stents and esophageal stents), stent grafts, vascular grafts, vascular access ports, embolization devices including cerebral aneurysm filler coils (including Guglilmi detachable coils and metal coils), myocardial plugs, pacemaker leads including drug plugs for pacing leads, left ventricular assist hearts and pumps, total artificial hearts, heart valves, vascular valves, tissue bulking devices, sutures, suture anchors, anastomosis clips and rings, tissue staples and ligating clips at surgical sites, cannulae, metal wire ligatures, orthopedic prosthesis, joint prostheses, as well as various other medical devices that are adapted for implantation or insertion into the body.

The medical devices of the present invention include implantable and insertable medical devices that are used for diagnosis, for systemic treatment, or for the localized treatment of any tissue or organ. Non-limiting examples are tumors; organs including the heart, coronary and peripheral vascular system (referred to overall as “the vasculature”), the urogenital system, including kidneys, bladder, urethra, ureters, prostate, vagina, uterus and ovaries, eyes, lungs, trachea, esophagus, intestines, stomach, brain, liver and pancreas, skeletal muscle, smooth muscle, breast, dermal tissue, cartilage, tooth and bone. As used herein, “treatment” refers to the prevention of a disease or condition, the reduction or elimination of symptoms associated with a disease or condition, or the substantial or complete elimination of a disease or condition. Typical subjects (also referred to as “patients”) are vertebrate subjects, more typically mammalian subjects and even more typically human subjects.

In some embodiments, the composite regions of the invention correspond to entire medical devices. In other embodiments, the composite regions correspond to one or more medical device portions. For instance, the composite regions can be in the form of one or more strands which are incorporated into a medical device, in the form of one or more layers formed over all or only a portion of an underlying medical device substrate, and so forth. Layers can be provided over an underlying substrate in a variety of locations, and in a variety of shapes (e.g., in desired patterns), and they can be formed from a variety of composite materials (e.g., different composite compositions may be provided at different locations). As used herein a “layer” of a given material is a region of that material whose thickness is small compared to both its length and width. As used herein a layer need not be planar, for example, taking on the contours of an underlying substrate. Layers can be discontinuous (e.g., patterned). Terms such as “film,” “layer” and “coating” may be used interchangeably herein.

Materials for use as underlying substrates include polymeric materials, ceramic materials and metallic materials, as well as other inorganic materials such as carbon- or silicon-based materials. For example, where a metallic substrate is employed, composite coatings in accordance with the present invention may provide improved interfacial adhesion vis-a-vis coatings with analogous polymeric coatings but which do not contain a ceramic component. Specific examples of metallic substrate materials may be selected, for example, from metals (e.g., biostable metals such as gold, platinum, palladium, iridium, osmium, rhodium, titanium, tantalum, tungsten, and ruthenium, and bioresorbable metals such as magnesium) and metal alloys, including metal alloys comprising iron and chromium (e.g., stainless steels, including platinum-enriched radiopaque stainless steel), alloys comprising nickel and titanium (e.g., Nitinol), alloys comprising cobalt and chromium, including alloys that comprise cobalt, chromium and iron (e.g., elgiloy alloys), alloys comprising nickel, cobalt and chromium (e.g., MP 35N), alloys comprising cobalt, chromium, tungsten and nickel (e.g., L605), and alloys comprising nickel and chromium (e.g., inconel alloys).

As used herein, “composite regions” are regions that contain a polymeric component and a ceramic component. As discussed further below, the polymeric and ceramic components may be associated with one another via covalent bonding and/or non-covalent interactions (e.g., van der Waals, polar-polar, nonpolar-nonpolar, ionic, etc.). Such regions may be porous or non-porous (e.g., when viewed under SEM).

In some embodiments of the invention, therapeutic agents are disposed within or beneath the composite regions, in which cases the composite regions may be referred to as carrier regions or barrier regions. By “composite carrier region” is meant a composite region which further comprises a therapeutic agent and from which the therapeutic agent is released. By “composite barrier region” is meant a composite region which is disposed between a source of therapeutic agent and a site of intended release, and which controls the rate at which therapeutic agent is released. For example, in some embodiments, the medical device consists of a composite barrier region that surrounds a source of therapeutic agent. In other embodiments, the composite barrier region is disposed over a source of therapeutic agent, which is in turn disposed over all or a portion of a medical device substrate.

As indicated above, the composite regions of the present invention contain or consist of (a) polymer component comprising a vinyl aromatic polymer and (b) a ceramic component comprising a metal or semi-metal oxide. For example, the composite regions may contain bi-continuous polymeric and ceramic phases, or one phase can be interspersed within the other (e.g., ceramic particles interspersed within one or more polymeric phase). For improved material properties, at least one of the phases may be of nanoscale dimension by which is meant that at least one cross-sectional dimension of the phase (e.g., the diameter for a spherical or cylindrical phase, the thickness for a ribbon- or plate-shaped phase, etc.) is less than 1 micron (1000 nm), for example, from 1000 nm to 300 nm to 100 nm to 30 nm to 10 nm or less in some embodiments. A decrease in such dimensions generally results in an increase in the interfacial area that exists between the polymeric and ceramic phases. Moreover, multiple polymeric and ceramic phases may be present. For example, multiple polymeric phases frequently exist where the composite region contains a block copolymer or a blend of different polymers.

Using techniques such as those described herein below, one can create a spectrum of composite materials ranging, for example, from a continuous polymeric phase with a ceramic phase incorporated at the molecular level, to a continuous polymeric phase with a dispersed nanoparticulate ceramic phase, to a bi-continuous system, to a continuous ceramic phase with a dispersed nanoparticulate polymeric phase. Consequently, one is provided with great latitude in tailoring the porosity of the composite regions of the present inventions. Among other effects, such changes in composition and porosity will affect the KDR of therapeutic agents that are disposed beneath or within the composites regions.

As used herein, “polymers” are molecules containing multiple copies (e.g., 5 to 10 to 25 to 50 to 100 to 250 to 500 to 1000 or more copies) of one or more constitutional units, commonly referred to as monomers.

A “vinyl aromatic polymer” is a polymer that contains one or more types of vinyl aromatic monomers as constitutional units.

Polymers may take on a number of configurations, which may be selected, for example, from cyclic, linear and branched configurations. Branched configurations include star-shaped configurations (e.g., configurations in which three or more chains emanate from a single branch point), comb configurations (e.g., configurations having a main chain and a plurality of side chains), dendritic configurations (e.g., arborescent and hyperbranched polymers), and so forth.

As used herein, “homopolymers” are polymers that contain multiple copies of a single constitutional unit. “Copolymers” are polymers that contain multiple copies of at least two dissimilar constitutional units, examples of which include random, statistical, gradient, periodic (e.g., alternating) and block copolymers.

As used herein, “block copolymers” are copolymers that contain two or more differing polymer blocks, for instance, because a constitutional unit (i.e., monomer) is found in one polymer block that is not found in another polymer block. As used herein, a “polymer block” is a grouping of constitutional units (e.g., 5 to 10 to 25 to 50 to 100 to 250 to 500 to 1000 or more units) that forms part or all of a polymer. Blocks can be branched or unbranched. Blocks can contain a single type of constitutional unit (also referred to herein as “homopolymeric blocks”) or multiple types of constitutional units (also referred to herein as “copolymeric blocks”) which may be provided, for example, in a random, statistical, gradient, or periodic (e.g., alternating) distribution. As used herein, a “chain” is a linear (unbranched) grouping of constitutional units (i.e., a linear block).

Specific examples of block copolymers for use in the invention include those which contain (a) one or more low glass transition temperature (T_{g}) polymer blocks and (b) one or more blocks containing one or more types of vinyl aromatic monomers (i.e., “vinyl aromatic blocks”), which are typically high T_{g} polymer blocks. Block copolymers having low and high T_{g} polymer blocks are known to possess many interesting physical properties due to the presence of a low T_{g} phase, which is soft and elastomeric at body temperature, and a high T_{g} phase, which is hard at body temperature. As used herein, “low T_{g} polymer blocks” are those that display a T_{g} that is below body temperature, for example, 37° C. to 20° C. to 0° C. to −25° C. to −50° C. or below. Conversely, as used herein, elevated or “high T_{g} polymer blocks” are those that display a glass transition temperature that is above body temperature, more typically 37° C. to 50° C. to 75° C. to 100° C. or above. T_{g} can be measured by various techniques including differential scanning calorimetry (DSC).

Block copolymer configurations may thus vary widely and include, for example, the following configurations (in which vinyl aromatic polymer chains are designated “V” and low T_{g} polymer chains are designated “L”), among others: (a) block copolymers having alternating chains of the type (VL)ₘ, L(VL)ₘ and V(LV)ₘ where m is a positive whole number of 1 or more, (b) multiarm copolymers such as X(LV)ₙ, and X(VL)ₙ, where n is a positive whole number of 2 or more, and X is a hub species (e.g., an initiator molecule residue, a residue of a molecule to which pre-formed polymer chains are attached, etc.), and (c) comb copolymers having an L chain backbone and multiple V side chains and vice versa (i.e., having a V chain backbone and multiple L side chains). It is conventional to disregard the presence of small entities such as hub species X in describing block copolymers, for example, with (VL)—X—(LV) and (LV)—X—(VL) being respectively designated as VLV and LVL triblock copolymers.

Specific examples of vinyl aromatic blocks include homopolymer and copolymer blocks containing one or more of the following vinyl aromatic monomers (listed along with published T_{g}'s for homopolymers of the same): (a) unsubstituted vinyl aromatic monomers, such as styrene (T_{g} 100° C.), and 2-vinyl naphthalene (T_{g} 151° C.), (b) vinyl substituted aromatic monomers such as α-methyl styrene, and (c) ring-substituted vinyl aromatic monomers including ring-alkylated vinyl aromatics such as 3-methylstyrene (T_{g} 97° C.), 4-methylstyrene (T_{g} 97° C.), 2,4-dimethylstyrene (T_{g} 112° C.), 2,5-dimethylstyrene (T_{g} 143° C.), 3,5-dimethylstyrene (T_{g} 104° C.), 2,4,6-trimethylstyrene (T_{g} 162° C.), and 4-tert-butylstyrene (T_{g} 127° C.), ring-alkoxylated vinyl aromatic monomers, such as 4-methoxystyrene (T_{g} 113° C.) and 4-ethoxystyrene (T_{g} 86° C.), ring-halogenated vinyl aromatic monomers such as 2-chlorostyrene (T_{g} 119° C.), 3-chlorostyrene (T_{g} 90° C.), 4-chlorostyrene (T_{g} 110° C.), 2,6-dichlorostyrene (T_{g} 167° C.), 4-bromostyrene (T_{g} 118° C.) and 4-fluorostyrene (T_{g} 95° C.) and ring-ester-substituted vinyl aromatic monomers such as 4-acetoxystyrene (T_{g} 116° C.), among others.

Specific examples of low T_{g} polymer blocks include homopolymer and copolymer blocks containing one or more of the following (listed along with published T_{g}'s for homopolymers of the same): (1) acrylic monomers including: (a) alkyl acrylates such as methyl acrylate (T_{g} 10° C.), ethyl acrylate (T_{g} −24° C.), propyl acrylate, isopropyl acrylate (T_{g} −11° C., isotactic), butyl acrylate (T_{g} −54° C.), sec-butyl acrylate (T_{g} −26° C.), isobutyl acrylate (T_{g} −24° C.), cyclohexyl acrylate (T_{g} 19° C.), 2-ethylhexyl acrylate (T_{g} −50° C.), dodecyl acrylate (T_{g} −3° C.) and hexadecyl acrylate (T_{g} 35° C.), (b) arylalkyl acrylates such as benzyl acrylate (T_{g} 6° C.), (c) alkoxyalkyl acrylates such as 2-ethoxyethyl acrylate (T_{g} −50° C.) and 2-methoxyethyl acrylate (T_{g} −50° C.), (d) halo-alkyl acrylates such as 2,2,2-trifluoroethyl acrylate (T_{g} −10° C.) and (e) cyano-alkyl acrylates such as 2-cyanoethyl acrylate (T_{g} 4° C.); (2) methacrylic monomers including (a) alkyl methacrylates such as butyl methacrylate (T_{g} 20° C.), hexyl methacrylate (T_{g} −5° C.), 2-ethylhexyl methacrylate (T_{g} −10° C.), octyl methacrylate (T_{g} −20° C.), dodecyl methacrylate (T_{g} −65° C.), hexadecyl methacrylate (T_{g} 15° C.) and octadecyl methacrylate (T_{g} −100° C.) and (b) aminoalkyl methacrylates such as diethylaminoethyl methacrylate (T_{g} 20° C.) and 2-tert-butyl-aminoethyl methacrylate (T_{g} 33° C.); (3) vinyl ether monomers including (a) alkyl vinyl ethers such as ethyl vinyl ether (T_{g} −43° C.), propyl vinyl ether (T_{g} −49° C.), butyl vinyl ether (T_{g} −55° C.), isobutyl vinyl ether (T_{g} −19° C.), 2-ethylhexyl vinyl ether (T_{g} −66° C.) and dodecyl vinyl ether (T_{g} −62° C.); (4) cyclic ether monomers include tetrahydrofuran (T_{g} −84° C.), trimethylene oxide (T_{g} −78° C.), ethylene oxide (T_{g} −66° C.), propylene oxide (T_{g} −75° C.), methyl glycidyl ether (T_{g} −62° C.), butyl glycidyl ether (T_{g} −79° C.), allyl glycidyl ether (T_{g} −78° C.), epibromohydrin (T_{g} −14° C.), epichlorohydrin (T_{g} −22° C.), 1,2-epoxybutane (T_{g} −70° C.), 1,2-epoxyoctane (T_{g} −67° C.) and 1,2-epoxydecane (T_{g} −70° C.); (5) ester monomers (other than acrylate and methacrylate esters) including ethylene malonate (T_{g} −29° C.), vinyl acetate (T_{g} 30° C.), and vinyl propionate (T_{g} 10° C.); (6) alkene monomers including ethylene, propylene (T_{g} −8 to −13° C.), isobutylene (T_{g} −73° C.), 1-butene (T_{g} −24° C.), 4-methyl pentene (T_{g} 29° C.), 1-octene (T_{g} −63° C.) and other olefins, trans-butadiene (T_{g} −58° C.), cis-isoprene (T_{g} −63° C.), and trans-isoprene (T_{g} −66° C.); (7) halogenated alkene monomers including vinylidene chloride (T_{g} −18° C.), vinylidene fluoride (T_{g} −40° C.), cis-chlorobutadiene (T_{g} −20° C.), and trans-chlorobutadiene (T_{g} −40° C.); and (8) siloxane monomers including dimethylsiloxane (T_{g} −127° C.), diethylsiloxane, methylethylsiloxane, methylphenylsiloxane (T_{g} −86° C.), and diphenylsiloxane, among others.

Specific examples of block copolymers include those containing one or more polyalkene blocks and one or more polystyrene blocks, for example, styrene-butadiene copolymers, styrene-ethylene-butylene copolymers (e.g., a polystyrene-b-poly(ethylene-co-butylene)-b-polystyrene (SEBS) triblock copolymer, available as Kraton® G series polymers), styrene-isoprene copolymers (e.g., polystyrene-polyisoprene-polystyrene triblock copolymer), and styrene-isobutylene copolymers (e.g., polyisobutylene-polystyrene diblock and polystyrene-polyisobutylene-polystyrene triblock copolymers such as those disclosed in U.S. Pat. No. 6,545,097 to Pinchuk).

As discussed in more detail below, hydrophilic derivatives of the above vinyl aromatic polymers, including ionomers and acidic and anhydride derivatives of the above, among others, are beneficial in various embodiments of the invention. The acidic derivates may be protonated (neutral) or deprotonated (anionic).

As indicated above, the ceramic component within the composite regions of the invention contain metal or semi-metal oxides, such as oxides of silicon, zirconium, titanium, aluminum, tin, hafnium, ruthenium, tantalum, molybdenum, tungsten, rhenium and/or iridium, among others.

The ceramic component within the composite regions of the invention may be formed using sol-gel techniques. In sol-gel techniques, the precursor materials used are typically inorganic metallic and semi-metallic salts, metallic and semi-metallic complexes/chelates (e.g., metal acetylacetonate complexes), metallic and semi-metallic hydroxides, or organometallic and organo-semi-metallic compounds (e.g., metal alkoxides and silicon alkoxides). Silicon alkoxides are beneficial due to the strength of the C—Si bond, which is stable with respect to hydrolysis, and because they can form a strong link between the polymeric and ceramic phases.

In a typical sol-gel process, precursor materials such as those above are subjected to hydrolysis and condensation (also referred to as polymerization) reactions, thereby forming a “sol”. For example, an alkoxide of choice (such as a methoxide, ethoxide, isopropoxide, n-propoxide, n-butoxide, isobutoxide, sec-butoxide, etc.) of a semi-metal or metal of choice (such as silicon, zirconium, titanium, aluminum, tin, hafnium, ruthenium, tantalum, molybdenum, tungsten, rhenium, iridium, etc.) may be dissolved in a suitable solvent, for example, in one or more alcohols. Subsequently, water or another aqueous solution, such as an acidic or basic aqueous solution (which aqueous solution can further contain organic solvent species such as alcohols) is added, causing hydrolysis and condensation to occur.

As can be seen from the simplified scheme below (from G. Kickelbick, “Concepts for the incorporation of inorganic building blocks into organic polymers on a nanoscale” Prog. Polym. Sci., 28 (2003) 83-114, the entire disclosure of which is incorporated herein by reference), the reaction is basically a ceramic network forming process in which the metal/semi-metal atoms (designated generally herein as M) within the ceramic phases are linked to one another via covalent linkages, such as M—O—M linkages, although other interactions are also commonly present including, for example, hydrogen bonding due to the presence of hydroxyl groups such as residual M—OH groups within the network:

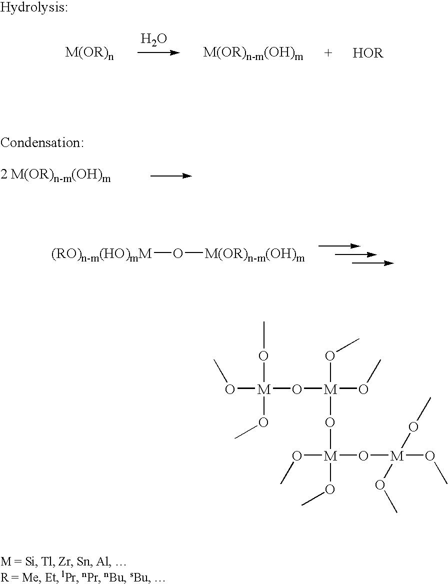

Further processing of the sol enables solid materials to be made in a variety of different forms. For instance, thin films can be produced on a substrate by spray coating, coating with an applicator (e.g., by roller or brush), spin-coating, dip-coating, and so forth, of the sol onto the substrate, whereby a “wet gel” is formed. Where dip coating is employed, the rate of withdrawal from the sol may be varied to influence the properties of the film. Monolithic wet gels can be formed, for example, by placing the sol into or onto a mold or another form (e.g., a sheet) from which the dried gel can be released. The wet gel is then dried. If the solvent in the wet gel is removed under supercritical conditions, a material commonly called an “aerogel” is obtained. If the gel is dried via freeze drying (lyophilization), the resulting material is commonly referred to as a “cryogel.” Drying at ambient temperature and ambient pressure leads to what is commonly referred to as a “xerogel.” Other drying possibilities are available including elevated temperature drying (e.g., in an oven), vacuum drying (e.g., at ambient or elevated temperatures), and so forth.

Using analogous processes, as well as principles of polymer synthesis, manipulation, processing, etc., composite materials for use in the present invention may be provided. Sol gel processes are suitable for use in conjunction with polymers and their precursors (as well as therapeutic agents, in some embodiments of the invention), for example, because they can be performed at ambient temperatures. A detailed review of various techniques for generating polymeric-ceramic composites can be found, for example, in Kickelbick, supra.

It is known, for example, to impregnate a gel such as a xerogel with monomer and polymerize the monomer within the gel.

Conversely, it is also known, for example, to generate polymeric-ceramic composites by conducting sol gel processing in the presence of a preformed polymer. Best results are often achieved where the polymer component has substantial non-covalent interactions with the ceramic component.

One way of improving the interactions between the polymeric and ceramic components is to employ a charged polymer (“ionomer”). For this purpose, polymers may be functionalized with anionic groups, such as sulfonate or carboxylate groups, among others, or cationic groups, such as ammonium groups, among others. Specific examples of vinyl aromatic ionomers include block copolymers having one or more sulfonated poly(vinyl aromatic) blocks and one or more polyalkene blocks, for example, sulfonated polystyrene-polyolefin-polystyrene triblock copolymers such as the sulfonated polystyrene-poly(ethylene/butylene)-polystyrene triblock copolymers described in U.S. Pat. No. 5,840,387, and sulfonated versions of the polystyrene-polyisobutylene-polystyrene (SIBS) triblock copolymers described in U.S. Pat. No. 6,545,097 to Pinchuk et al. and the poly[(styrene-co-p-methylstyrene)-b-isobutylene-b-(styrene-co-p-methylstyrene)] triblock copolymers described in S. J. Taylor et al., Polymer 45 (2004) 4719-4730, which polymers may be sulfonated, for example, using the processes described in U.S. Pat. No. 5,840,387 and U.S. Pat. No. 5,468,574, among other sufonated block copolymers. Sulfonated polymers are also described in Elabd and Napadensky, “Sulfonation and Characterization of Poly(styrene-isobutylene-styrene) Triblock Copolymers at High Ion-Exchange Capacities,” Polymer 45 (2004) 3037-3043; Elabd et al., Journal of Membrane Sci., 217 (2003) 227; Blackwell and Mauritz, Polymer 45 (2004) 3457, Storey and Baugh, Polymer 42 (2001) 2321; Edmonson and Fontanella, Solid State Ionics 152-153 (2002) 355; and Kwon and Puskas, European Polymer Journal 40 (2004) 119.

For example, ionomers may be formed as described in K. A. Mauritz et al., Polymer 43 (2002) 4315-4323 and K. A. Mauritz et al., Polymer 43 (2002) 5949-5958. Briefly, sulfonic acid groups (—SO₃H) of a partially sulfonated polystyrene-polyisobutylene-polystyrene block copolymer are converted to sulfate form by neutralization of these groups with a base such as sodium hydroxide, tetrabutylammonium hydroxide (TBAH) or benzyltrimethylammonium (BTMA) hydroxide, thereby forming the ionomeric form of the polymer.

Mauritz et al. further demonstrated that the distinct phase separated morphology of this block co-polymer ionomer may be used as a morphological template for sol-gel reactions involving the tetraethylorthosilicate (TEOS) monomer, thereby creating a novel polymeric-ceramic composite material. In this work, they combined (a) a polystyrene domain-selective swelling solvent, such as dimethylacetamide (DMAc) (DMAc was chosen to selectively swell the ionic polystyrene domains in the ionomeric block copolymer based on the fact that the solvent will dissolve polystyrene homopolymer, but not polyisobutylene homopolymer) with (b) large organic counterions such as benzyltrimethylammonium for the sulfonated styrene blocks, which were found to result in a higher degree of order as compared to smaller counterions such as Na⁺. The swollen polymer was then immersed in a sol-gel reactive solution, for example, a solution of TEOS, DMAc, and acidified water. Mauritz et al. theorized that these measures facilitated preferential migration of tetraethoxy silicate (TEOS) and hydrolyzed TEOS monomers, i.e., (EtO)₄₋ₓSi(OH)ₓ where x is an integer of 1 to 4, along the ionic polystyrene regions where the condensation reaction took place. Regardless of theory, this combination resulted in a so-called “template” nanocomposite morphology.

As used herein “large counterions” include those having van der Waals volumes greater than 50 Å³, for example from 50 Å³ to 100 Å³ to 250 Å³ to 500 Å³ or more. van der Waals volume may be calculated, for example, as described in M. Ue et al., “A Convenient Method to Estimate Ion Size for Electrolyte Materials Design,” Journal of The Electrochemical Society, 149 (10) A 1385-A1388 (2002).

Mauritz et al. have also conducted sol-gel processing in which all of the molecular building blocks for forming the nanocomposite are supplied in a single composition and dried to form materials with heterogeneous nanostructured morphologies. For example, nanocomposites based on sulfonated polystyrene-b-polyisobutylene-b-sulfonated polystyrene (sSIBS) and sulfonated polystyrene-b-[ethylene-co-butylene]-b-sulfonated polystyrene (sSEBS) have been formed from formulations in which sulfonated block copolymer, cosolvent, TEOS and water are present, which are subsequently dried. See, e.g., K. A. Mauritz et al., “Self-assembled organic/inorganic hybrids as membrane materials,” Electrochimica Acta 50 (2004) 565-569 and K. A. Mauritz et al. “Viscoelastic properties and morphology of sulfonated poly(styrene-b-ethylene/butylene-b-styrene) block copolymers (sBCP), and sBCP/[silicate] nanostructured materials,” Polymer 45 (2004) 3001-3016.

Another way of improving the interactions between the polymer and the ceramic components is to create covalent bonds between them. This result can be achieved via a number of known techniques, including the following: (a) providing species with both polymeric and ceramic precursor groups and thereafter conducting polymerization and hydrolysis/condensation simultaneously, (b) providing a ceramic sol with polymer precursor groups (e.g., groups that are capable of participation in a polymerization reaction, such as vinyl groups or cyclic ether groups) and thereafter conducting an organic polymerization step, (c) providing polymers with reactive groups (e.g., at the polymer ends, along the polymer backbone, etc.) that are capable of participation in hydrolysis/condensation reactions (e.g., metal or semi-metal alkoxide groups, maleic anhydride groups, etc.).

An example of the latter approach is set forth in C- S Wu and H- T Liao, “Modification of Polyethylene-Octene Elastomer by Silica Through a Sol-Gel Process,” Journal of Applied Polymer Science, Vol. 88, 966-972 (2003), who describe a process in which inorganic-organic hybrid structures are formed by dissolving TEOS, H₂O and HCl in THF, which is then added to a polymer melt of maleic anhydride-grafted polyethylene-octene elastomer. Thus, in this instance, the sol-gel precursors (e.g., alkoxy compound, H₂O and catalyst) are added to a polymer melt.

A maleated derivative of SEBS (m-SEBS), which has the same combination of styrene and maleic anhydride functional groups, is commercially available as Kraton® FG 1901X.

m-SEBS may also be sulfonated as described in S. K. Ghosh et al., “Physicomechanical and Dielectric Properties of Magnesium and Barium Ionomers Based on Sulfonated Maleated Styrene-Ethylene/Butylene-Styrene Block Copolymer,” Journal of Applied Polymer Science, Vol. 77, 816-825 (2000) to from sulfonated maleated SEBS (s-m-SEBS). In this regard, T. Kwee et al., “Poly[styrene-b-maleated (ethylene/butylene)-b-styrene] (mSEBS) block copolymers and mSEBS/inorganic nanocomposites: I. Morphology and FTIR characterization,” Polymer 46 (2005) 3871-3883 describe a process in which mSEBS (commercially available as Kraton® FG1901X) is sulfonated and then dissolved in tetrahydrofuran (THF) solvent. FTIR spectra indicated that a mixture of open and closed anhydride rings were present in the sulfonated mSEBS. Nanocomposites were prepared from a multicomponent solution which contained the sulfonated mSEBS, the THF solvent, H₂O, an alkoxysilane (e.g., TEOS, phenyltriethoxysilane, or isobutyltrimethoxysilane), and an n-propanol co-solvent. The multicomponent solution was allowed to react for several hours, after which the resultant sol-gel-reactive solution was cast, dried and annealed to form various mSEBS/inorganic nanocomposites. As indicated above, the presence of a ceramic component in the coatings of the invention may allow for improved adhesion to substrates, particularly metallic substrates. See also P -C Chiang et al., “Effects of titania content and plasma treatment on the interfacial adhesion mechanism of nano titania-hybridized polyimide and copper system,” Polymer 45 (2004) 4465-4472. The presence of maleic anhydride functional groups will also allow for increased adhesion to metallic surfaces. See, e.g., Handbook of Adhesives, Irving Skeist, ed., 2nd Edition, New York: van Nostrand Reinhold, 1977, p. 335, and “Reactive functional copolymers,” Copyright 2006, SpecialChem S.A.

Numerous techniques are thus available for providing composite regions for medical devices in accordance with the present invention.

For example, various techniques described above involve the formation of a suspension (e.g., a “sol”) or melt containing a ceramic component and a polymer component. Alternatively, a composite material may be preformed which has thermoplastic characteristic, in which case it may be heated to form a melt for further processing. Such suspensions or melts may be applied to a substrate to form a composite region. The substrate can correspond, for example, to all or a portion of an implantable or insertable medical device (e.g., a stent, balloon, or guide wire, among many others) to which the suspension or melt is applied. The substrate can also correspond, for example, to a template, such as a mold, from which the composite region is removed after solidification. In other embodiments, for example, extrusion and co-extrusion techniques, composite regions for medical devices are formed without the aid of a substrate.

Specific examples of techniques for processing suspensions and melts include molding, casting, extrusion and coating techniques such as injection molding, blow molding, solvent casting, extrusion into sheets, fibers, rods, tubes and other cross-sectional profiles of various lengths, screen printing, ink jet printing, dip coating, spin coating, spray coating, coating with an applicator (e.g., by roller or brush), web coating, and techniques involving coating via mechanical suspension including air suspension. The ionomeric/polar nature of many of the polymers used in accordance with the invention, makes them suitable for deposition processes based on electrical phenomena such as electrospray and electrophoresis processes, among others.

Other techniques involve first forming a polymeric region, followed by the introduction of a ceramic precursor to the polymeric region. This polymeric region may be formed from a solution or melt of the polymer using techniques such as those describe above, with the resulting polymeric region corresponding, for example, to a medical device, a medical device component, a medical device coating, etc. As discussed above, interactions between the polymeric region and the ceramic precursors may be enhanced, for example, by employing ionomers with large counterions and/or by employing a solvent to swell the polymeric region.

In certain embodiments, the composite regions of the present invention contain one or more therapeutic agents. “Therapeutic agents,” “biologically active agents,” “drugs,” “pharmaceutically active agents,” “pharmaceutically active materials,” and other related terms may be used interchangeably herein and include genetic therapeutic agents, non-genetic therapeutic agents and cells. A wide variety of therapeutic agents can be employed in conjunction with the present invention. Numerous therapeutic agents are described here.

Suitable non-genetic therapeutic agents for use in connection with the present invention may be selected, for example, from one or more of the following: (a) anti-thrombotic agents such as heparin, heparin derivatives, urokinase, and PPack (dextrophenylalanine proline arginine chloromethylketone); (b) anti-inflammatory agents such as dexamethasone, prednisolone, corticosterone, budesonide, estrogen, sulfasalazine and mesalamine; (c) antineoplastic/antiproliferative/anti-miotic agents such as paclitaxel, 5-fluorouracil, cisplatin, vinblastine, vincristine, epothilones, endostatin, angiostatin, angiopeptin, monoclonal antibodies capable of blocking smooth muscle cell proliferation, and thymidine kinase inhibitors; (d) anesthetic agents such as lidocaine, bupivacaine and ropivacaine; (e) anti-coagulants such as D-Phe-Pro-Arg chloromethyl ketone, an RGD peptide-containing compound, heparin, hirudin, antithrombin compounds, platelet receptor antagonists, anti-thrombin antibodies, anti-platelet receptor antibodies, aspirin, prostaglandin inhibitors, platelet inhibitors and tick antiplatelet peptides; (f) vascular cell growth promoters such as growth factors, transcriptional activators, and translational promotors; (g) vascular cell growth inhibitors such as growth factor inhibitors, growth factor receptor antagonists, transcriptional repressors, translational repressors, replication inhibitors, inhibitory antibodies, antibodies directed against growth factors, bifunctional molecules consisting of a growth factor and a cytotoxin, bifunctional molecules consisting of an antibody and a cytotoxin; (h) protein kinase and tyrosine kinase inhibitors (e.g., tyrphostins, genistein, quinoxalines); (i) prostacyclin analogs; (j) cholesterol-lowering agents; (k) angiopoietins; (l) antimicrobial agents such as triclosan, cephalosporins, antimicrobial peptides such as magainins, aminoglycosides and nitrofurantoin; (m) cytotoxic agents, cytostatic agents and cell proliferation affectors; (n) vasodilating agents; (o)agents that interfere with endogenous vasoactive mechanisms, (p) inhibitors of leukocyte recruitment, such as monoclonal antibodies; (q) cytokines; (r) hormones; (s) inhibitors of HSP 90 protein (i.e., Heat Shock Protein, which is a molecular chaperone or housekeeping protein and is needed for the stability and function of other client proteins/signal transduction proteins responsible for growth and survival of cells) including geldanamycin, (t) beta-blockers, (u) bARKct inhibitors, (v) phospholamban inhibitors, (w) Serca 2 gene/protein, (x) immune response modifiers including aminoquizolines, for instance, imidazoquinolines such as resiquimod and imiquimod, (y) human apolioproteins (e.g., AI, AII, AIII, AIV, AV, etc.).

Specific examples of non-genetic therapeutic agents, not necessarily exclusive of those above, include paclitaxel (including particulate forms thereof, for instance, protein-bound paclitaxel particles such as albumin-bound paclitaxel nanoparticles, e.g., ABRAXANE), sirolimus, everolimus, tacrolimus, Epo D, dexamethasone, estradiol, halofuginone, cilostazole, geldanamycin, ABT-578 (Abbott Laboratories), trapidil, liprostin, Actinomcin D, Resten-NG, Ap-17, abciximab, clopidogrel, Ridogrel, beta-blockers, bARKct inhibitors, phospholamban inhibitors, Serca 2 gene/protein, imiquimod, human apolioproteins (e.g., AI-AV), growth factors (e.g., VEGF-2), as well a derivatives of the forgoing, among others.

Exemplary genetic therapeutic agents for use in connection with the present invention include anti-sense DNA and RNA as well as DNA coding for: (a) anti-sense RNA, (b) tRNA or rRNA to replace defective or deficient endogenous molecules, (c) angiogenic factors including growth factors such as acidic and basic fibroblast growth factors, vascular endothelial growth factor, epidermal growth factor, transforming growth factor α and β, platelet-derived endothelial growth factor, platelet-derived growth factor, tumor necrosis factor α, hepatocyte growth factor and insulin-like growth factor, (d) cell cycle inhibitors including CD inhibitors, and (e) thymidine kinase (“TK”) and other agents useful for interfering with cell proliferation. Also of interest is DNA encoding for the family of bone morphogenic proteins (“BMP's”), including BMP-2, BMP-3, BMP-4, BMP-5, BMP-6 (Vgr-1), BMP-7 (OP-1), BMP-8, BMP-9, BMP-10, BMP-11, BMP-12, BMP-13, BMP-14, BMP-15, and BMP-16. Currently preferred BMP's are any of BMP-2, BMP-3, BMP-4, BMP-5, BMP-6 and BMP-7. These dimeric proteins can be provided as homodimers, heterodimers, or combinations thereof, alone or together with other molecules. Alternatively, or in addition, molecules capable of inducing an upstream or downstream effect of a BMP can be provided. Such molecules include any of the “hedgehog” proteins, or the DNA's encoding them.

Vectors for delivery of genetic therapeutic agents include viral vectors such as adenoviruses, gutted adenoviruses, adeno-associated virus, retroviruses, alpha virus (Semliki Forest, Sindbis, etc.), lentiviruses, herpes simplex virus, replication competent viruses (e.g., ONYX-015) and hybrid vectors; and non-viral vectors such as artificial chromosomes and mini-chromosomes, plasmid DNA vectors (e.g., pCOR), cationic polymers (e.g., polyethyleneimine, polyethyleneimine (PEI)), graft copolymers (e.g., polyether-PEI and polyethylene oxide-PEI), neutral polymers PVP, SP1017 (SUPRATEK), lipids such as cationic lipids, liposomes, lipoplexes, nanoparticles, or microparticles, with and without targeting sequences such as the protein transduction domain (PTD).

Cells for use in connection with the present invention include cells of human origin (autologous or allogeneic), including whole bone marrow, bone marrow derived mono-nuclear cells, progenitor cells (e.g., endothelial progenitor cells), stem cells (e.g., mesenchymal, hematopoietic, neuronal), pluripotent stem cells, fibroblasts, myoblasts, satellite cells, pericytes, cardiomyocytes, skeletal myocytes or macrophage, or from an animal, bacterial or fungal source (xenogeneic), which can be genetically engineered, if desired, to deliver proteins of interest.

Numerous therapeutic agents, not necessarily exclusive of those listed above, have been identified as candidates for vascular treatment regimens, for example, as agents targeting restenosis. Such agents are useful for the practice of the present invention and suitable examples may be selected from one or more of the following: (a) Ca-channel blockers including benzothiazapines such as diltiazem and clentiazem, dihydropyridines such as nifedipine, amlodipine and nicardapine, and phenylalkylamines such as verapamil, (b) serotonin pathway modulators including: 5-HT antagonists such as ketanserin and naftidrofuryl, as well as 5-HT uptake inhibitors such as fluoxetine, (c) cyclic nucleotide pathway agents including phosphodiesterase inhibitors such as cilostazole and dipyridamole, adenylate/Guanylate cyclase stimulants such as forskolin, as well as adenosine analogs, (d) catecholamine modulators including α-antagonists such as prazosin and bunazosine, β-antagonists such as propranolol and α/β-antagonists such as labetalol and carvedilol, (e) endothelin receptor antagonists, (f) nitric oxide donors/releasing molecules including organic nitrates/nitrites such as nitroglycerin, isosorbide dinitrate and amyl nitrite, inorganic nitroso compounds such as sodium nitroprusside, sydnonimines such as molsidomine and linsidomine, nonoates such as diazenium diolates and NO adducts of alkanediamines, S-nitroso compounds including low molecular weight compounds (e.g., S-nitroso derivatives of captopril, glutathione and N-acetyl penicillamine) and high molecular weight compounds (e.g., S-nitroso derivatives of proteins, peptides, oligosaccharides, polysaccharides, synthetic polymers/oligomers and natural polymers/oligomers), as well as C-nitroso-compounds, O-nitroso-compounds, N-nitroso-compounds and L-arginine, (g) Angiotensin Converting Enzyme (ACE) inhibitors such as cilazapril, fosinopril and enalapril, (h) ATII-receptor antagonists such as saralasin and losartin, (i) platelet adhesion inhibitors such as albumin and polyethylene oxide, (j) platelet aggregation inhibitors including cilostazole, aspirin and thienopyridine (ticlopidine, clopidogrel) and GP IIb/IIIa inhibitors such as abciximab, epitifibatide and tirofiban, (k) coagulation pathway modulators including heparinoids such as heparin, low molecular weight heparin, dextran sulfate and β-cyclodextrin tetradecasulfate, thrombin inhibitors such as hirudin, hirulog, PPACK(D-phe-L-propyl-L-arg-chloromethylketone) and argatroban, FXa inhibitors such as antistatin and TAP (tick anticoagulant peptide), Vitamin K inhibitors such as warfarin, as well as activated protein C, (l) cyclooxygenase pathway inhibitors such as aspirin, ibuprofen, flurbiprofen, indomethacin and sulfinpyrazone, (m) natural and synthetic corticosteroids such as dexamethasone, prednisolone, methprednisolone and hydrocortisone, (n) lipoxygenase pathway inhibitors such as nordihydroguairetic acid and caffeic acid, (o) leukotriene receptor antagonists, (p) antagonists of E- and P-selectins, (q) inhibitors of VCAM-1 and ICAM-1 interactions, (r) prostaglandins and analogs thereof including prostaglandins such as PGE1 and PGI2 and prostacyclin analogs such as ciprostene, epoprostenol, carbacyclin, iloprost and beraprost, (s) macrophage activation preventers including bisphosphonates, (t) HMG-CoA reductase inhibitors such as lovastatin, pravastatin, fluvastatin, simvastatin and cerivastatin, (u) fish oils and omega-3-fatty acids, (v) free-radical scavengers/antioxidants such as probucol, vitamins C and E, ebselen, trans-retinoic acid and SOD mimics, (w) agents affecting various growth factors including FGF pathway agents such as bFGF antibodies and chimeric fusion proteins, PDGF receptor antagonists such as trapidil, IGF pathway agents including somatostatin analogs such as angiopeptin and ocreotide, TGF-β pathway agents such as polyanionic agents (heparin, fucoidin), decorin, and TGF-β antibodies, EGF pathway agents such as EGF antibodies, receptor antagonists and chimeric fusion proteins, TNF-α pathway agents such as thalidomide and analogs thereof, Thromboxane A2 (TXA2) pathway modulators such as sulotroban, vapiprost, dazoxiben and ridogrel, as well as protein tyrosine kinase inhibitors such as tyrphostin, genistein and quinoxaline derivatives, (x) MMP pathway inhibitors such as marimastat, ilomastat and metastat, (y) cell motility inhibitors such as cytochalasin B, (z) antiproliferative/antineoplastic agents including antimetabolites such as purine analogs (e.g., 6-mercaptopurine or cladribine, which is a chlorinated purine nucleoside analog), pyrimidine analogs (e.g., cytarabine and 5-fluorouracil) and methotrexate, nitrogen mustards, alkyl sulfonates, ethylenimines, antibiotics (e.g., daunorubicin, doxorubicin), nitrosoureas, cisplatin, agents affecting microtubule dynamics (e.g., vinblastine, vincristine, colchicine, Epo D, paclitaxel and epothilone), caspase activators, proteasome inhibitors, angiogenesis inhibitors (e.g., endostatin, angiostatin and squalamine), rapamycin, cerivastatin, flavopiridol and suramin, (aa) matrix deposition/organization pathway inhibitors such as halofuginone or other quinazolinone derivatives and tranilast, (bb) endothelialization facilitators such as VEGF and RGD peptide, and (cc) blood rheology modulators such as pentoxifylline.

Numerous additional therapeutic agents useful for the practice of the present invention are also disclosed in U.S. Pat. No. 5,733,925 assigned to NeoRx Corporation, the entire disclosure of which is incorporated by reference.

Various techniques may be employed in incorporating the therapeutic agent(s) into the composite regions of the present invention.

For instance, therapeutic agent(s) may be incorporated by exposing them to previously formed composite regions. For instance, a fluid containing dissolved or dispersed therapeutic agent may be contacted with the composite regions by dipping, spraying, coating with an applicator (e.g., by roller or brush), spin-coating, web coating, techniques involving coating via mechanical suspension including air suspension, ink jet techniques, and combinations of these processes, among other techniques. Water, organic solvents, subcritical fluids, critical point fluids, supercritical fluids, and so forth can be used as carriers for the therapeutic agent.

In other instances, therapeutic agent(s) may be incorporated into the composite regions concurrently with their formation. For example, where the composite region is cast from a single formulation (e.g., a solution, suspension, melt, etc.) containing all of the molecular elements required for the formation of the composite region, the therapeutic agent(s) may be added to that formulation.

As another example, where the ceramic precursors are introduced into a previously formed polymer region (e.g., a medical device coating, etc.), the therapeutic agent(s) may be combined with the ceramic precursors, or it may be introduced into the polymer prior to introduction of the ceramic precursors.

For instance, as noted above, ionomers containing sulfonated vinyl aromatic groups have been used to form various composite regions. These ionomers may be formed by converting the sulfonic acid groups (—SO₃H) of sulfonated polymers to sulfate form by neutralization of these groups with a base such as sodium hydroxide, TBAH or BTMA hydroxide, thereby forming the ionomeric form of the polymer.

A basic therapeutic agent may be employed for this purpose, resulting in the distribution of therapeutic counterions along the polymer backbone. Furthermore, in many instances, cations of therapeutic argents are large and thus analogous to benzyltrimethylammonium cations, which were found by Mauritz et al. to be beneficial for composite formation.

Examples of such therapeutic agents include the free base forms of the following cisplatins, among others:

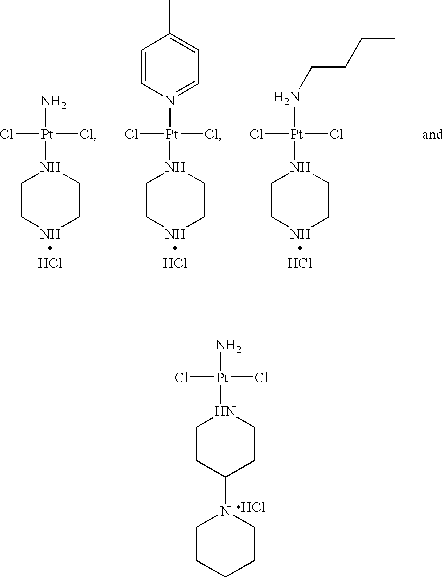

which are described in P.A. Nguewa et al., “Water soluble cationic trans-platinum complexes which induce programmed cell death in the protozoan parasite Leishmania infantum,” Journal of Inorganic Biochemistry 99 (2005) 727-736. Further specific examples include paclitaxel derivatives such as the free based form of paclitaxel N-methyl pyridinium mesylate. See, e.g., U.S. Pat. No. 6,730,699; Duncan et al., Journal of Controlled Release 74 (2001)135; Duncan, Nature Reviews/Drug Discovery, Vol. 2, May 2003, 347; Jaber G. Qasem et al, AAPS PharmSciTech 2003, 4(2) Article 21. In addition to these, U.S. Pat. No. 6,730,699, also describes various forms of paclitaxel in which paclitaxel is conjugated to basic polymers including poly(l-lysine), poly(d-lysine), and poly(dl-lysine), among others. Cisplatins and taxanes such as paclitaxel are known to have antineoplastic/antiproliferative/anti-miotic activity.

Analogous reactions may be employed in which basic therapeutic agents are used to neutralize further acidic polymers beyond polymers with sulfonic acid groups, such as those containing carboxyl groups, among others, and in which acidic therapeutic agents are used to neutralize basic polymers, such as those containing amino groups, among others.

Therapeutic agents may also be covalently linked to the ceramic and/or polymeric components of the composite regions of the present invention. As noted above, maleic anhydride derived polymers have proven useful in forming organic-inorganic composite regions. It is also known to covalently bind amine groups containing therapeutic agents with maleic anhydride derived polymers. See e.g., J. Hoste et al., “Polymeric prodrugs,” International Journal of Pharmaceutics 277 (2004) 119-131, which describe work in which a prodrug is formed by the reaction of poly(styrene-co-maleic acid/anhydride) (SMA) with amino groups found in the antitumor protein neocarcinostatin (NCS). This drug has been successfully introduced into clinical practice for cancer therapy.

Using analogous techniques, NCS and other amine-group-containing therapeutic agents may be attached to maleic anhydride derived vinyl aromatic polymers either before or after formation of the ceramic component. One example of a maleic anhydride derived vinyl aromatic polymer among many others is m-SEBS, described above, which has the same combination of styrene and maleic anhydride functional groups as SMA. Another example is sulfonated mSEBS, described in T. Kwee et al., supra. They report that FTIR spectra suggest that a mixture of open and closed anhydride rings are present in the sulfonated mSEBS, suggesting the suitability of this polymer for covalent binding.

As noted above, composite barrier regions are provided over therapeutic-agent-containing regions in some embodiments of the invention. In these embodiments, a composite region can be formed over a therapeutic-agent-containing region, for example, using one of the suspension- or melt-based techniques described above. Alternatively, a previously formed composite region may be adhered over a therapeutic agent containing region.

### EXAMPLE

Sulfinated SIBS (sSIBS) is dissolved/activated/swollen in an appropriate solvent or combination of solvents such as DMAc, Toluene, THF, or a combination thereof. A suitable precursor solution is then added under stirring to the sSIBS solution. Precursor solutions are prepared by dissolving a metal alkoxide, such as titanium tetraisopropoxide, or an alkoxy silane, such as TEOS, aminopropyltrimethoxysilane or chloropropyltrimethoxysilane, in a suitable solvent or solvent mixture such as methanol, ethanol, butanol, toluene or a combination thereof. Then, distilled water and a catalyst such as an acid are added in the appropriate volume and concentration to initiate hydrolysis. A paclitaxel solution in ethanol or another suitable organic solvent is added before or immediately after the addition of the water and catalyst. The solution is stirred under suitable processing conditions until the hydrolysis and condensation reactions are advanced to the desired degree (usually several hours). The resulting sol is cast, extruded, applied through various coating processes, etc. to obtain a desired form, which is dried in an oven for aging.

Although various embodiments are specifically illustrated and described herein, it will be appreciated that modifications and variations of the present invention are covered by the above teachings and are within the purview of the appended claims without departing from the spirit and intended scope of the invention.

1. A medical device comprising a composite region that comprises (a) a non-extruded polymeric component comprising a vinyl aromatic polymer that comprises polar groups, ionic groups, or both and (b) a ceramic component comprising a metal oxide, semi-metal oxide, or both, wherein said medical device is an implantable or insertable medical device.

2. The medical device of claim 1, wherein said medical device is selected from a stent, a stent graft, a balloon, a guide wire, a vena cava filter, a cerebral aneurysm filler coil, and a pacemaker lead.

3. The medical device of claim 1, wherein said composite region is a coating that is disposed over an underlying medical device.

4. The medical device of claim 1, wherein said underlying medical device is a metallic medical device.

5. The medical device of claim I, comprising a plurality of said composite regions.

6. The medical device of claim 1, wherein said composite region comprises nanoscale ceramic particles.

7. The medical device of claim 1, wherein said ceramic component comprises an oxide selected from oxides of silicon, titanium, zirconium, aluminum, tin, tantalum, iridium, ruthenium, and combinations thereof.

8. The medical device of claim 1, wherein said semi-metal is silicon.

9. The medical device of claim 1, wherein a plurality of polymeric phases are present in said composite region.

10. The medical device of claim 1, wherein the vinyl aromatic polymer is a copolymer comprising a vinyl aromatic monomer and an alkene monomer.

11. The medical device of claim 1, wherein the vinyl aromatic polymer is a block copolymer comprising (a) a polymer block that comprises a vinyl aromatic monomer and (b) a low T_{g} polymer block.

12. The medical device of claim 11, wherein said low T_{g} polymer block comprises an alkene monomer.

13. The medical device of claim 1, wherein said vinyl aromatic polymer is an ionomer.

14. The medical device of claim 13, wherein said ionomer is a sulfonated vinyl aromatic polymer.

15. The medical device of claim 1, wherein said sulfonated vinyl aromatic polymer is selected from sulfonated polystyrene-b-poly(ethylene-co-butylene)-b-polystyrene and sulfonated polystyrene-b-polyisobutylene-b-polystyrene.

16. The medical device of claim 1, wherein said ionomer comprises a multi-atom molecular counterion having a van der Waals volume greater than 50 Å³.

17. The medical device of claim I, wherein the polymeric component and the ceramic component are covalently linked.

18. The medical device of claim 1, wherein the polymeric component and the ceramic component are covalently linked through an —O— linkage.

19. The medical device of claim 1, wherein the vinyl aromatic polymer comprises a maleic anhydride residue.

20. The medical device of claim 1, wherein the vinyl aromatic polymer comprises a plurality of maleic anhydride residues along its length.

21. The medical device of claim 1, further comprising a therapeutic agent disposed within or beneath said composite region.

22. The medical device of claim 21, wherein said therapeutic agent is an anti-proliferative agent.

23. The medical device of claim 21, wherein said therapeutic agent is ionic.

24. The medical device of claim 23, wherein said vinyl aromatic polymer is an ionomer and said ionic therapeutic agent provides counterions for said ionomer.

25. The medical device of claim 23, wherein said vinyl aromatic polymer comprises a plurality of sulfate groups along its length and wherein said ionic therapeutic agent provides countercations for said sulfate groups.

26. The medical device of claim 21, wherein said therapeutic agent is covalently bound to said vinyl aromatic polymer.

27. The medical device of claim 26, wherein said vinyl aromatic polymer comprises a plurality of maleic anhydride residues along its length and wherein said therapeutic agent is covalently bound to said vinyl aromatic polymer through said maleic anhydride residues.

28. The medical device of claim 21, wherein said therapeutic agent is selected from taxanes, cisplatins, and antitumor proteins.

29. The medical device of claim 26, wherein said vinyl aromatic polymer comprises a plurality of sulfate groups and a plurality of maleic anhydride residues along its length.

30. The medical device of claim 26, wherein said vinyl aromatic polymer is selected from sulfonated maleated polystyrene-b-poly(ethylene-co-butylene)-b-polystyrene and sulfonated maleated polystyrene-b-polyisobutylene-b-polystyrene.

31. The medical device of claim 1, wherein said composite region is created by a process that comprises (a) providing a suspension that comprises said vinyl aromatic polymer and ceramic precursors selected from metal alkoxides, silicon-alkoxides and combinations thereof; and (b) removing water from said suspension.

32. The medical device of claim 31, wherein said composite region is created by a process that comprises (a) applying a layer of said suspension to a substrate and (b) removing water from said layer.

33. The medical device of claim 31, wherein said suspension comprises a block co-polymer ionomer which acts as a morphological template for formation of said ceramic component.

34. The medical device of claim 1, wherein the composite region is in the form of a therapeutic-agent-containing plug.

35. The medical device of claim 1, wherein the vinyl aromatic polymer comprises protonated acidic groups, deprotonated acidic groups, acid anhydride groups, or a combination thereof.

36. The medical device of claim 1, wherein the composite region is non-porous.
